(19) United States
(12) Patent Application Publication
(10) Pub. No.: US 2022/0152434 A1
(43) Pub. Date: May 19, 2022

(54) FACE MASK

(71) Applicant: Johannes Hendrikus Ninaber, s-Hertogenbosch (NL)

(72) Inventor: Johannes Hendrikus Ninaber, s-Hertogenbosch (NL)

(21) Appl. No.: 17/333,515

(22) Filed: May 28, 2021

(30) Foreign Application Priority Data
May 29, 2020 (NL) 2025713

(51) Int. Cl. A62B 23/02; A62B 9/06; A61F 9/02; A62B 9/04; A41D 13/11

(52) U.S. Cl. CPC A62B 23/025; A62B 9/06; A41D 13/11; A62B 9/04; A61F 9/029

(57) ABSTRACT

A face mask comprising a front part (1) and a back part (4), wherein the back part (4) is beak-shaped and the front part (1) is installed to match the outline of the back part (4) and a bracket (3), that forms a pliable rigid arc starting at jaw level on one cheek in front of or over the nose, is installed to form an arc leading to the other side of the jaw, the front part (1) is applied slightly stretched over the outline of the bracket (3), and a filter is enclosed between the front part and the bracket.

### BACKGROUND AND SUMMARY OF THE INVENTION

Face masks are made of fabric or a hard shell-like material and both designs come with some disadvantages.

A rigid or hard-shell mask encloses the jaw and the top of the nose in a bowl.

Speech is impeded by the jaw being held together with the top of the nose through the edge of the hard, rigid mask. This causes an abrasive effect when moving one's jaw to talk.

A disadvantage of fabric masks or masks made from other elastic materials is that they have to be pulled tautly over the face in order for them to closely fit around the jaw and nose. This flattens the nose and leaves marks on the face. When wearing such a mask, the taut fabric in front of the mouth is in contact with the lips and impedes free mouth movement when talking. The lips then rub against the fabric when talking.

The invention provides a fabric face mask in which the disadvantages have been overcome.

The face mask according to the invention comprises a bracket made of a pliable material. The bracket keeps the mask clear of the mouth so that speaking is easier, unlike conventional fabric masks where the fabric of the mask is pulled against the mouth.

The mask is largely made of a flexible fabric material and the shape is such that the mouth can move freely to make talking easier.

In general, the material of fabric masks has a certain flexibility whereas filter materials are usually rigid, since otherwise the mesh size of the filter would not be fixed. This makes it particularly difficult to incorporate a filter material into a fabric mask.

### Various Designs are Known:

International Patent Application WO2017/083289 discloses a respirator including a mask body having an upper panel, a lower panel, and a central panel disposed between the upper and lower panels. The mask body can also include a left attachment mechanism disposed between a left tab of the mask body and a left portion of at least one of the upper panel and the lower panel when the mask is in an open condition, and a right attachment mechanism disposed between a right tab of the mask body and a right portion of at least one of the upper panel and the lower panel when the mask body is in the open condition.

International Patent Application WO2019/069061 discloses a fold flat personal respiratory device comprising: a generally planar central panel including a layer of filter media and having opposing first and second side edges and first and second end edges; a first and second side panel joined to said central panel along the first and second side edge; the device manually configurable from a folded configuration in which the side panels are substantially parallel to a planar surface of the central panel and an operational configuration in which the central and first and second side panels form a cup-shaped respiratory chamber; the device further comprises a rigid or semi-rigid endoskeleton member mounted along a planar surface of the central panel and extending between the first and second side edges and/or the first and second end edges.

International Patent Application WO 2009/048748 discloses a filtering face-piece respirator comprising a mask body and a harness. The mask body includes (i) a filtering structure, (ii) a plastic support structure, and (iii) a nose clip that is secured to the plastic support structure by being molded therein.

Korean Patent Application KR 2016/0084714 discloses a respiratory frame for masks. A frame to facilitate respiration is fixated on a portion of a nose and a mouth of a mask fabric close to a respiratory holes. The frame including a nose protrusion part and a chin protrusion part formed thereon is interposed and fixed on an inner lining of fabric closed to the respiratory holes, preventing the fabric from adhering to the nose and the mouth and thus facilitating respiration.

Korean Patent Application KR 2019/0002982 discloses a yellow dust mask that changes according to the UV concentration, and in particular, when wearing a yellow dust mask and going out, the color changes according to the UV concentration.

European Patent Application EP0894443 discloses a facial mask with a easy handling in piling up during the transportation or storage, having the rear side of the mask main body portion not in direct contact with the user's mouth in use. A plastic thin plate is a narrow flat plate with a rectangular cross-section. It is arranged inside a mask main body portion linearly along the folded portions with the longer sides of the cross-section contacted to the fabric of the mask main body portion. Further, the both ends are fixed to the mask main body portion, with the substantially center part fixed to the rear side of the mask main body portion.

The present invention provides a face mask in which the flexible qualities of a fabric material are combined with the advantages of a rigid material. The face mask according to the invention comprises a front part and a back part, wherein the back part is beak-shaped and the front part has been attached the back part and the front part and back part together form an envelope and a bracket has been provided in the envelope, which bracket comprises a pliable rigid arc starting at jaw level on one cheek over the nose and extending in an arc to the other side of the jaw and the front part being slightly stretched and tautly applied along the outline of the bracket.

The bracket causes the mask material to hang in front of the mouth and making it easy to talk, because the moving mouth is hardly in contact with the fabric applied over it.

In an embodiment according to the invention, there is a filter behind the front part. The bracket has been placed behind the filter inside the front part and causes the edges of the front part to be tensioned along the edge of the bracket such that they are slightly stretched and that they are not tautly applied over the nose and mouth.

The filter is preferably of a virtually identical shape as the line of attachment of the front part to the back part along the same outline in the upper half, except for the side where the mask attachment engages. This causes the non-stretch filter to be enclosed between the front part and the bracket.

The fact that the front part is applied slightly stretched along the outline of the bracket causes the seam between an edge of the filter and an edge of the front part to be sealed preventing any air from escaping through the filter here. If the filter had been inserted between the front and back parts without the bracket an unstable filter might deform, possibly causing gaps through which air can pass the filter without being filtered.

The front part is preferably made of a highly air permeable material to enable maximum air flow through the filter, facilitating breathing.

The front part is attached to the back part by, for example, stitching, gluing, fusing or melting. In an embodiment of the present invention, the bracket may be integrated with the aforementioned attachment.

The back part is preferably made of a non-air permeable material and forms a closed fit with the face in a curve from the underside of the jaw over the cheeks along the top of the nose bridge and back over the other cheek to the chin.

A nose bracket is provided in the back part between the filter and the face that can follow the face contours in order to seal the edge formed between the back part and the face.

The pliable nose bracket contributes to the sealing of the face mask at the eye sockets and nose in order to prevent air leakage or at least greatly reduce the probability of air leakage.

In an embodiment of the present invention, the bracket of pliable rigid material is made of plastic of preferably between 0.5 and 2 mm, for example about 1 mm thick. The plastic material of that thickness inserted between the parts of the mask bends in an arc from cheek to cheek and in front of the nose because it has inherent surface rigidity.

In an embodiment of the invention, the back part which creates the seal with the face is formed from one part for example by cutting or punching it entirely from airtight fabric. The back part and the front part together form an envelope because the outer edges of the front and back parts are connected together at least at the upper and lower sides. The envelope is open through a slot in the back part and the inside is accessible through this opening in the back part. The filter can be installed between the front and back parts through this opening. The filter is preferably slightly oversized but would bend and deform if it were enclosed in the envelope because filter materials cannot stretch and do not have sufficient stiffness and body to ensure that they will retain their shape when inside the envelope. The bracket is placed in the envelope. The shape of the filter along its upper edge is virtually identical to that of the bracket. The interconnected air-permeable front part and airtight back part stretch sufficiently whereby the bracket gives the filter its shape whilst being enclosed together in the envelope formed by the front and back parts.

The bracket is larger at the tip of the nose to prevent any edges of the material from rubbing against the tip of the nose.

The filter is preferably non-slip or rough and remains in the seam at the underside of the envelope allowing the filter to slip behind the material of the bracket and between the front part. In an embodiment, the lower edge of the filter is attached to the front part or the back part.

### BRIEF DESCRIPTION OF THE DRAWINGS

These and further aspects of the invention are described below and illustrated by means of the drawings:

In the drawings:

FIG. 1 shows a face mask tautly stretched over a face;

FIG. 2 shows the face mask of FIG. 1 in more detail in a layer-by-layer view.

FIG. 3 shows a detail of a face mask according to the invention;

FIG. 4 shows a front part, back part, bracket and filter in their unassembled state;

FIGS. 5a and 5b show a side view and a top view of a bracket;

FIG. 6 shows how to apply a face mask;

FIG. 7 shows a view through the front part with the mouth open;

FIG. 8 shows another embodiment of the face mask according to the invention;

FIG. 9 shows another embodiment of the face mask according to the invention;

FIG. 10 shows another embodiment of the face mask according to the invention.

The figures have not always been drawn to scale; as a rule, like numerals denote like elements.

### DETAILED DESCRIPTION

The parts of the face mask shown in the figures are referred to with the following numerals:
1 Front part:
   1a. Lower stitching
   1b. Upper edge
   1c. Buckle stitching
   1d. Buckle attachment.
2 Filter:
   2a. Lower edge
   2b. Upper edge
   2c. Edge with buckle.
3 Bracket:
   3a. Lower protrusion
   3b. Upper edge
   3c. Buckle contact
   3d. Nose impact
   3e. Splashguard.
4 Back part:
   4a. Lower inside seam
   4b. Upper edge
   4c. Edge with buckle
   4d. Buckle attachment
   4e. Nose bridge position
   4f. Finger lug
   4g. Corner of the mouth
   4h. Opening
   4i. Nose covering part.
5 Nose bridge:
   5a. Nose bridge.
6 Buckle
   6a. Hole in the buckle.
7 Straps
   7a. Overhead strap
   7b. Neck strap.

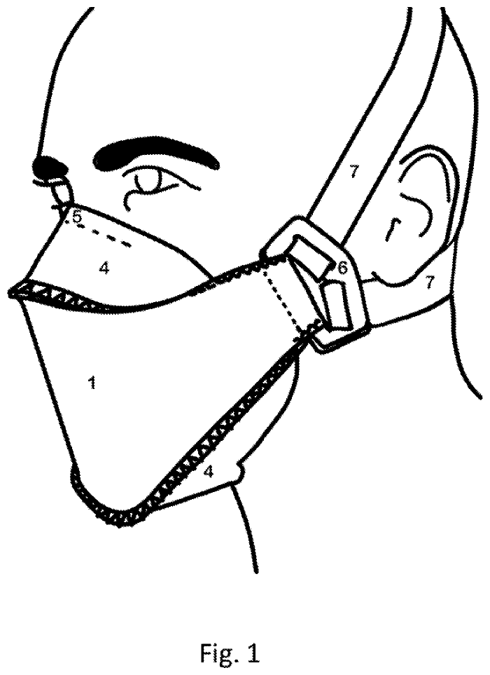

FIG. 1 shows a face mask applied over a face with straps (7) along the back of the skull. The face mask comprises a front part (1) that is attached to a back part (4). The back part (4) preferably, but not exclusively, comprises a nose bridge (5). The face mask preferably comprises a buckle (6) and preferably elastic straps (7) to secure the face mask to the head and allow it to be worn.

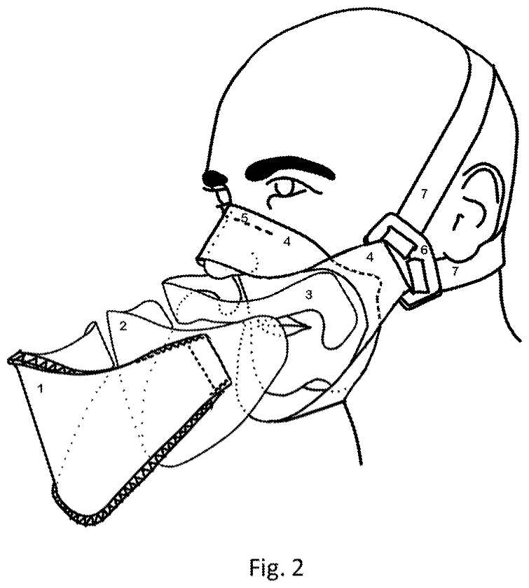

FIG. 2 shows a more detailed view of the face mask of FIG. 1. The back part (4) comprises an upper part and a lower part, that combine to display a beak shape. The beak shape opening of the back part (4) is attached to the front part (1) such that the outlines match. Furthermore, the face mask comprises a filter (2) and a bracket (3). Bracket (3) forms a pliable rigid arc that extends almost vertically in front of the face. One end of bracket (3) is located at jaw level on one cheek, the bracket (3) continues to the other side of the chin over, or in front of, the nose, forming an arc. The front part (1) is slightly stretched and tautly applied over the outline of the bracket (3). A filter (2) has been installed between the front part (1) and the bracket (3). In this embodiment, the shape of the filter (2) is virtually identical to the enclosed space created between the front part (1) and the back part (4) with the same outlines. Straps 7 may be made of elastic and are also referred to below as “elastic band(s)”.

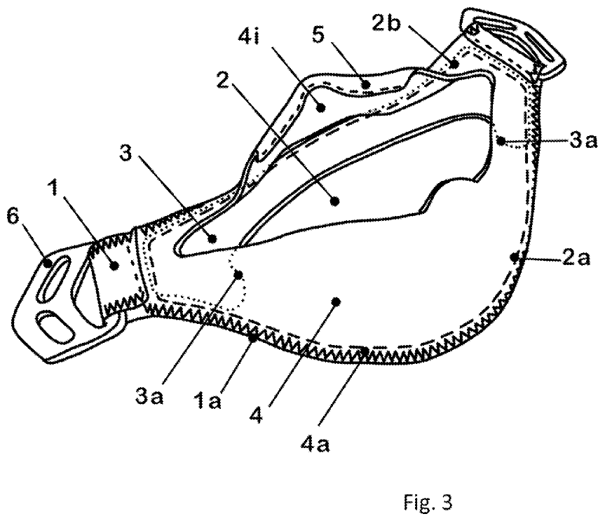

FIG. 3 shows a mask from a view to the back part (4) installed along the lower edge (4a) with the lower edge (1a) of the front part (1) and the element covering the inner side of the nose (4i) of the back part (4), and it shows the position of the pliable nose bridge (5) on the inner side of the back part (4), with the bracket (3) and filter (2) being visible through the cut-out in the back part (4) and dotted lines showing how they continue to the two buckles. The figure also shows how the loop in the front part (1) is connected to the back part (4) by the buckle (6). The nose bridge (5) is malleable in order to enable it to be adjusted such that the convexity of the nose and the connection to the underside of the eye sockets under the eyes are sealed.

The nose bridge (5) is the malleable part made of malleable material, for example, and preferably of, malleable metal.

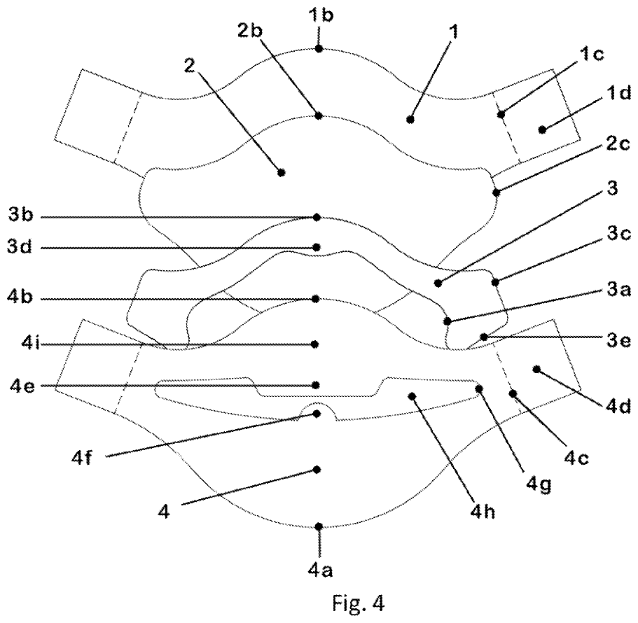

FIG. 4 shows the front part (1), back part (4), bracket (3) and filter (2) in their unassembled flat state.

There is a filter (2) between the bracket (3) and the front part (1). The shape of the filter (2) is approximately the same as that of the front part (1).

In an embodiment according to the invention, the front part (1) may itself consist of a filtering material such that an additional filter (2) is not necessary. The front part (1) and filter (2) then form one whole. Embodiments can comprise a combination of a filtering front part (1) and an additional filter (2), for example, a front part with a coarse filtering effect and an additional finer filter.

In this example, the front part (1) comprises the following elements:
1a. Lower stitching for attachment to the back part (4).
1b. Upper edge running from one side to the opposite side, passing over the nose in the center.
1c. Buckle stitching defining the ends of the envelope created with the back part on both sides.
1d. Buckle attachment loop created by including extra material beyond the buckle stitching, folding this into a loop and stitching it into a tube-shaped loop into which a buckle can be enclosed.

In this embodiment, the filter (2) comprises the following elements:
2a. Lower edge. This extends from the underside of the buckle stitching on one cheek to the connection with the underside of the buckle stitching on that overlying side of the face and passes in front of, or partly under, the tip of the jaw and along the line of the jaw to the other cheek. The dimensions along these lines are equal to, or preferably greater than, this line created with the front part in a non-stretched state, so that the filter is always equal to, or bigger than, the envelope space.
2b. Upper edge. This is the seam that follows the attachment of the front and back parts in the envelope line with the connection to the top of the buckle seam and that—due to the attachment of the front and back parts—extends forward over the cheek and in front of the nose to the opposite cheek where it connects to the top of the buckle seam on that side. If the front part is not tensioned, this seam is identical to the available space.
2c. Buckle connection side. This forms the outer edge of the filter connecting the end of the upper edge to the end of the lower edge on the same side.

In this example, bracket (3) comprises the following elements:
3a. Lower protruding part. This forms the connection of the bracket to the lower edge of the envelope that continues partly towards the front of the jaw and thus creates a firm and stable position when the bracket is enclosed in the partially stretched envelope and prevents the shape of the bracket from sagging downwards.
3b. Upper edge. This forms the edge that follows the bracket to seamlessly connect in the line that is created by the front and back parts having been connected together along their tops and forming a seam that follows the shape of the line in a flat plane and thus remains identical, even in a curved plane.
3c. Buckle contact side. This is the outer edge of the bracket that connects the end of the upper edge to the end of the lower edge on the same side and along which the bracket is pressed towards the buckle by stretch tension on the front and back parts and which pressure causes the bracket to become convex.
3d. Nose impact. This is the position in the centre of the flat plane of the bracket that, when installed, is directly in front of the nose and has a partly enlarged surface area so that the lower edge of this part under the nose falls outside the area that contacts the nose.

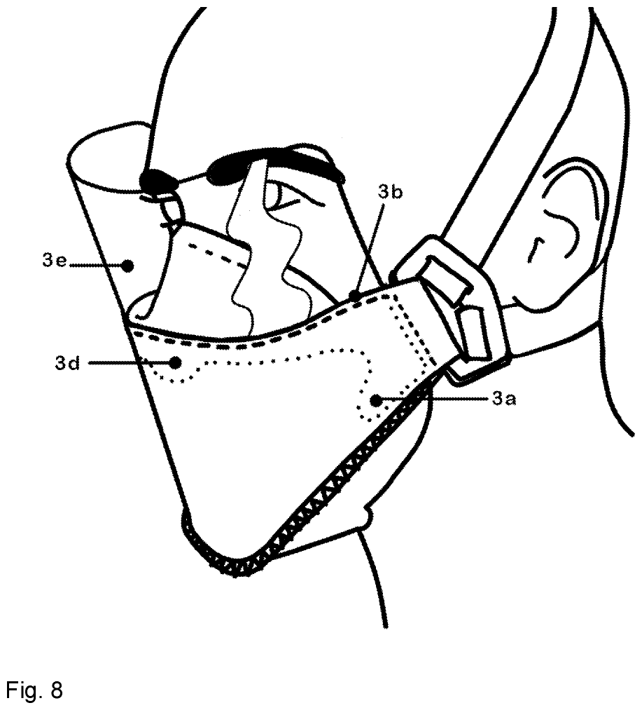

The bracket (3) can be provided with a splashguard (3e, see FIG. 8) to protect the eyes as a part of the bracket or as an element that can be installed separately.

In this embodiment, the back part (4) comprises the following elements:
4a. Lower inner seam for connection to the front part.
4b. Upper edge running from one side to the opposite side, passing over the nose in the center.
4c. Edge with buckle defining the ends of the envelope created with the front part on both sides.
4d. Buckle attachment loop created by including extra material beyond the buckle stitching, folding this into a loop and stitching it into a tube-shaped loop into which a buckle can be enclosed.
4e. Nose bridge position is a part that is folded-over and stitched or glued, and which, when it was a part of the flat cut-out of the back part, was part of the material opened up as a face opening and which houses a pliable element enabling the strap part to be more easily formed into a position that is not closed in forming the fit of the mask with the face.
4f. Finger lug is a part of the material that, when it was a part of the flat cut-out of the back part, was part of the material opened up as a face opening and enables the part of the panel that extends under the jaw to be grasped easily by fingers and to be positioned properly under the chin
4g. Corner of the mouth is a rounded element on the outside of the cut-out in the back part where the round shape of the cut-out eliminates the probability of the flat line of the opening tearing if it were cut straight and ended in a sharp angle
4h. Opening as an element and consequence of the cut-out in the back part that gives the mouth, jaw and nose access to the back part and which element has been shaped such that its shape, combined with the partial flexibility of the materials used, offers sufficient space to ensure a good fit around these facial elements.
4i. Nose covering part is the part of the back part that is visible in the face view and extends below the eye across the nose to below the other eye.

The back part (4) can be opened and the lower and upper part will then become beak-shaped, the opening (4h) forms the opening of the beak shape.

The front part (1) is attached to the back part (4) by gluing, for example, or, as is the case here, by stitching.

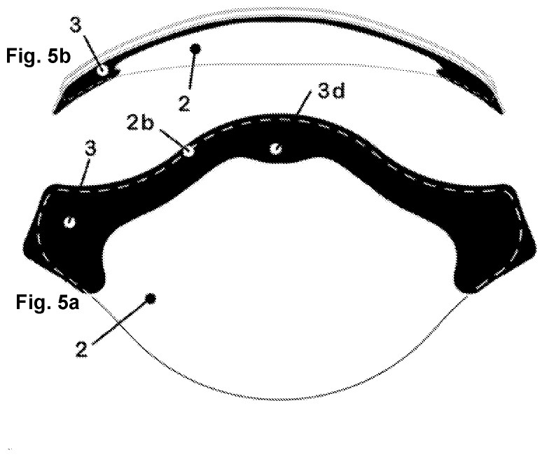

FIG. 5a shows a flat view in which the filter (2) has been placed behind the bracket (3) and the continuation of the shape of the filter (2) in bracket (3) is shown as a dotted line and it is visible that the filter (2) fits within the shape along the upper side and is virtually flush with the upper edge (3b) of the bracket (3).

FIG. 5b shows a similar composition as in FIG. 5a showing that the filter (2) seamlessly connects to the bracket (3) when the bracket (3) becomes cylindrically convex due to tension caused by stretching in the panel sections (1 and 4) and a second filter connects along the same line.

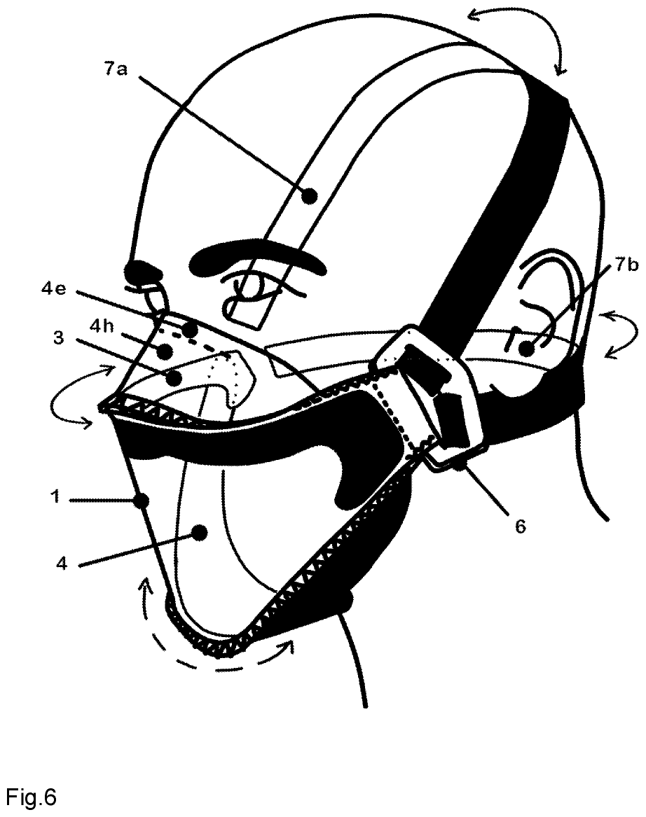

FIG. 6 shows an embodiment according to the invention in which it is visible that the bracket (3) and the elastic band (7b) form a circle around the head with the buckle (6) acting as the connecting element and where the tensile forces on the back part (4) caused by tensioning the elastic band (7b and 7a) are evenly distributed along and over the bracket (3) which, due to the convexity in its shape, offers room for the back part (4h) that covers the space between the bracket (3) and the face and is largely free from tensile stress due to the tensioning of the elastic band (7b) along the underside of the head.

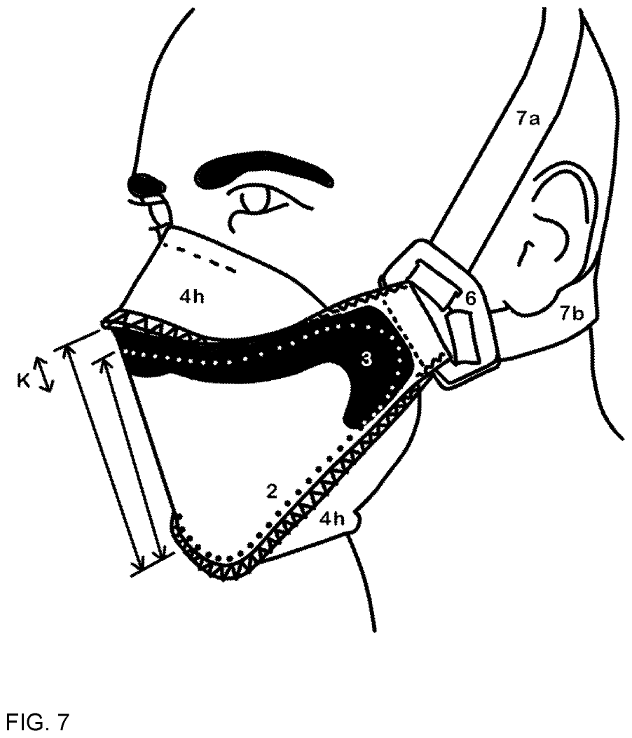

FIG. 7 shows the position of the filter (2) as a dotted line in front of the bracket (3) wherein upward and downward movement of the back part causes part (4h) under the jaw along the filter (2) to move along, creating an opening above the edge of the filter (2b) that is sealed because the bracket (3) with the upper edge (3b) of the bracket remains sealed and enclosed due to the tension caused by stretching the material of the front part (1) and the back part (4) and prevents air from entering or escaping past the filter (2) through space k when the mouth is stretched open. This prevents any unfiltered air from entering or being emitted through this opening.

FIG. 8 shows an embodiment according to the invention wherein the bracket (3) is mounted as a fixed part preferably, but not exclusively, along the upper edge (3b) between the front part (1) and the back part (4) in such a way that the bracket (3) extends to at least the upper side with a splashguard (3e) that protects the eyes as a transparent screen.

It will be clear that the invention enables many variations and that the invention is not limited to the examples described above.

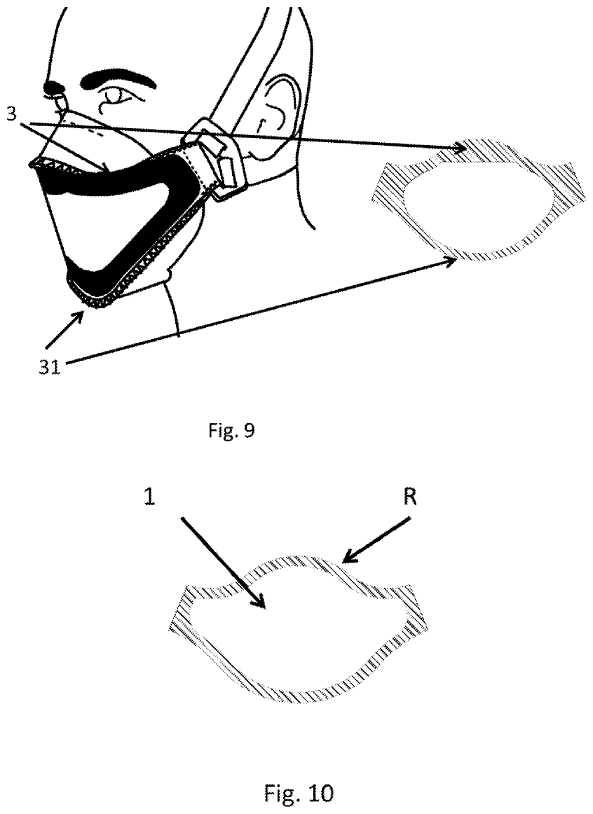

FIG. 9 shows another embodiment of the face mask according to the invention. In this embodiment, the face mask comprises a lower bracket (31) in addition to the bracket (3). The lower bracket (31) follows the jaw line along the outside of the jaw. Preferably, as shown in FIG. 9, bracket (3) and lower bracket (31) form a whole and together form a closed shape. The material of the face mask is then kept away from the mouth. The lower bracket (31) follows the outside of the jaw line and there is preferably no material of the lower bracket (31) under the jaw that might block or hinder the jaw movement.

FIG. 10 shows a detail of another embodiment of the face mask according to the invention. The material of the front part has been compacted along edge R. The compacted part of the front part, which is not or hardly air-permeable, is attached to the back part. This improves sealing and reduces the probability of air leakage. The compacted edge R, which, as is clear by comparing FIGS. 9 and 10 has the same shape, and position as the bracket 3 in FIG. 9, and may form in the embodiment of FIG. 10 the bracket.

The assembly of front and back part may be manufactured separately. The invention is also embodied in a face mask comprising a front part and a back part, wherein the back part is, when worn on a face, beak-shaped and comprises a cut-out part forming a face opening and the front part is attached to the back part some distance in front of the face.

I claim:

1. A face mask comprising
a front part,
a back part, and
a bracket at least partly in between the front part and back part,
wherein:
the back part is, when worn on a face, beak-shaped and comprises a cut-out part forming a face opening, and
the front part is attached to the back part some distance in front of the face,
the bracket comprises a pliable arc starting at jaw level on one cheek over the nose and extending in an arc to the other side of the jaw, and
the front part is slightly stretched by the bracket.

2. The face mask of claim 1, wherein:
one or more filters are located behind the front part, and
the bracket is positioned behind the filter or filters within an envelope formed by front part and back part to enclose the filter or filters.

3. The face mask of claim 2, wherein the shape of the filter or filters along an upper contour is virtually identical in shape to that of the front part along the same outline.

4. The face mask of claim 1, wherein the front part is made of highly air permeable material.

5. The face mask of claim 1, wherein the back part is made of a single piece of non-air permeable material and has a cut-out shape that forms a closed fit with the face over the nose and mouth in a connection from the lower jaw over the cheeks along the upper nose and back over the other cheek to the connection with the lower side of the chin.

6. The face mask of claim 1, wherein the back part is made of a single piece.

7. The face mask of claim 1, wherein the bracket has been punched or cut out of a sheet of material.

8. The face mask of claim 1, wherein leakage of air past the substantially dimensionally stable filter is blocked by the bracket that moves along with the flexibility of the stretchable front part.

9. The face mask of claim 1, wherein:
the back part comprises a nose bridge at an edge of the cut-out part, and
the nose bridge is made of a malleable material.

10. The face mask of claim 1, wherein the front part and/or back part are held in position in a partially stretched form, in the upper half only, by the bracket.

11. The face mask of claim 10, wherein the bracket is given a certain arched convexity in front of the face due to tension caused by stretching the material of the front part and/or the back part.

12. The face mask of claim 1, wherein:
the back part is beak-shaped by means of a cut-out in the material and
the front part shows an outline that is virtually identical to that of the back part,
the front part connects and is attached to the back part, thereby creating an envelope with a closed outline that extends along the upper edge and the bracket between the connection of the front part and the back part outside the envelope that is created,
the front part extends above the mask as a splashguard for the eyes, and
the bracket is situated between the front part and the back part with a pliable rigid arc starting at jaw level on one cheek and continuing in an almost vertical plane in front of the nose in an arc to the other side of the jaw which has thus been slightly stretched and applied along the upper edge of the bracket and attached.

13. The face mask of claim 1, wherein front part comprises a filtering material.

14. The face mask of claim 1, wherein
the face mask comprises a lower bracket
wherein the lower bracket follows the jawline along the outside of the lower jaw.

15. The face mask of claim 14 wherein bracket and lower bracket form a whole.

16. A face mask comprising:
a front part and
a back part,
wherein the back part is, when worn on a face, is beak-shaped and comprises a cut-out part forming a face opening and
the front part is attached to the back part at a distance in front of the face.

17. The face mask of claim 16 wherein the back part is made of a single piece of non-air permeable material and has a cut-out shape that forms a closed fit with the face over the nose and mouth in a connection from the lower jaw over the cheeks along the upper nose and back over the other cheek to the connection with the lower side of the chin.
